(19) United States
(12) Patent Application Publication
(10) Pub. No.: US 2004/0143191 A1
(43) Pub. Date: Jul. 22, 2004

(54) Device for noninvasive measurement of the blood pressure, in particular for the continuous monitoring of ambulatory blood pressure for an ambulatory patient

(76) Inventor: Yves Faisandier, Paris (FR)

Correspondence Address: Robert M. Isackson, Orrick, Herrington & Sutcliffe LLP, 666 Fifth Avenue, New York, NY, 10103, US

(21) Appl. No.: 10/724,520

(22) Filed: Nov. 26, 2003

(30) Foreign Application Priority Data
Nov. 29, 2002 (FR) 02 15069

(51) Int. Cl. A61B 5/02

(52) U.S. Cl. 600/485; 600/586

(57) ABSTRACT

A device for noninvasive measurement of blood pressure, in particular for continuous monitoring of blood pressure for an ambulatory patient. This device includes at least one sensor, placed on the thoracic wall of a patient, that is able to transform the acoustic signals generated by the closing of the cardiac valves and transmitted through the thorax into an electronic phonocardiographic signal. The phonocardiographic signal is processed to discriminate and extract a vibratory profile (18) related to the cardiac noise periodically produced at the end of the systole. At least one predetermined parameter of the vibratory profile is analyzed, in particular the amplitude (20) separating the extrema from the signal, and according to this parameter, a value of the phono-arterial index representative of the blood pressure is delivered.

### FIELD OF THE INVENTION

The present invention relates to a device for noninvasive measurement of blood pressure, intended in particular for the continuous follow-up of an ambulatory patient.

### BACKGROUND OF THE INVENTION

Several techniques are known to measure a patient's blood pressure. A first technique, which allows an uninterrupted measurement, introduces an intra-arterial catheter connected to a pressure sensor. This technique, which is by nature invasive, is used mainly in intensive care units, surgery or during certain explorations. Its invasive character and the risk of hemorrhage makes it, however, unusable in practice in an ambulatory patient context.

Another technique, which is noninvasive and commonly used in ambulatory practice, equips the patient with a periodically inflated arm-band. The indication of the blood pressure is then given with the arm-band inflated, either by measurement of the Korotkov noises (namely, the noise emitted by the artery when its pressure oscillates on both sides of the pressure of the balloon) or by the Pachon technique (i.e., the measurement of the variations of volume of the artery by a second balloon placed downstream from the first). This technique provides specific measurements, and by automating the measurements it is possible to obtain blood pressure values over certain intervals of time in order to reconstruct a pressure curve having a duration of several hours to several days.

However, this technique also presents a certain number of disadvantages.. First of all, the periodic inflating of the balloon disturbs the patient and can wake one who is sleeping. In addition, as the pressure is not continuously measured, there remains a risk that a hypertensive or hypotensive crisis will not be detected if the crisis occurs between two measurements. Lastly, it is difficult to imagine an ambulatory follow-up of long duration, that is one lasting several days, because the repetitive compression of the tissues of the member inserted in the balloon is intolerable for the patient.

A third measurement technique, also of a noninvasive nature, uses a balloon placed around a member (in general a finger) and permanently inflated with a pressure enslaved to (i.e., varies as a function of) the volume of internal blood. The blood volume is then measured using infra-red light passing through the finger. One thus obtains a continuous curve of the blood volume change which is correlated to the blood pressure. This technique can be miniaturized in order to be implemented in an ambulatory practices. To carry out monitoring over periods of several hours, it is necessary, however, to envisage frequently changing the position of the balloon because the tissues do not support a permanent or sustained compression. A suggested solution is to use two balloons, each one on a different finger, and alternate which balloon is used. The complexity of the implementation of this technique and its discomfort for the patient, however, limit the current use of this technique and its generalization for an ambulatory follow-up, in particular over a long duration such as a complete day or even several consecutive days.

### OBJECTS AND SUMMARY OF THE INVENTION

It is, therefore, an object of the present invention to propose a new technique for measuring blood pressure, one that is noninvasive by nature and delivers a continuous, sustainable blood pressure measurement.

It is a further object of the present invention to provide such a measurement that does not cause discomfort to the patient, and allows use in an ambulatory practice without the known disadvantages, including interference with sleep, and over durations of analysis that can reach several consecutive days.

To this end, one aspect of the present invention is directed to a device having: at least one sensor that is able to be placed on the thoracic wall of a patient, and able to detect the acoustic signals generated by the closing of the cardiac valves and transmitted through the thorax, and produce an electronic phonocardiographic signal representing such acoustic signals; a discriminating means, able to recognize and extract from the phonocardiographic signal a vibratory profile related to the cardiac noise periodically produced at the end of the systole, often referred to as the “second” cardiac noise; and analyzing means, able to analyze at least one predetermined parameter of the vibratory profile and to deliver in response, according to the at least one parameter, a phono-arterial index value representative of the blood pressure.

Preferably, the discriminating means and the analyzing means can operate in real time on the phonocardiographic signal as it is procured. In an alternative embodiment, the discriminating means and the analyzing means can operate after acquisition of the phonocardiographic signal, where the signal is memorized (stored in a memory) beforehand by a means for recording the phonocardiographic signal. The signal may be recorded as raw acquired data or as pretreated data (e.g., raw data that has been filtered, conditioned and preferably digitized).

The aforementioned at least one parameter can be a parameter selected from among the group including, for example, the amplitude separating the extrema of the phonocardiographic signal over the duration of the profile, the energy of this signal, the variation of the derivative of this signal, the surface of this signal, and a combination of more than one of the foregoing parameters.

In a preferred embodiment, the analyzing means operates to apply to the aforementioned at least one parameter a weighted value, which can vary from one profile to the next profile. More particularly, the discriminating means also is able to recognize and extract from the phonocardiographic signal a second vibratory profile, related to the cardiac noise that is periodically produced at the beginning of systole (typically referred to as the first cardiac noise). The analyzing means is then able to process the at least one predetermined parameter belonging to the second vibratory profile and produce therefrom a weighted value.

In yet another embodiment, the device can include at least two sensors, with a circuit means designed to combine the two signals delivered by these sensors into a single signal, e.g., an average, that in turn is applied to the discriminating means.

Preferably, the device can include means for evaluating the body position of the patient, as well as a self-learning means that is able to memorize beforehand a plurality of average levels of the phonocardiographic signal according to a like plurality of corresponding body positions, and in which the analyzing means comprise means to apply to the aforementioned parameter(s) a weighted value, as a function of the average level previously memorized for the body position detected at the moment of the analysis.

In a more preferred embodiment, the device also includes a low-pass filter for low-pass filtering the phono-arterial index. In particular, it can be envisaged to include a means for evaluating the respiratory frequency, such that the low-pass filtering can be implemented as means for adaptive filtering at a variable cut-off frequency, according to the value of the respiratory frequency at the time of the analysis. In the alternative, the device can include means for analyzing the respiratory cycle as well as means for filtering the phono-arterial index, these means of filtering preferably being a means for dynamic filtering at a variable gain, according to the phase of the respiratory cycle at the time of the analysis.

In an advantageous embodiment of the present invention, when the phonocardiographic signal is treated in real time, the device also includes additional means for measuring the blood pressure that is able to deliver an absolute value of blood pressure measurement. This other means of measurement is then activated or inhibited in a selective manner according to the value of phono-arterial index delivered by the analyzing means.

Also in the case of the real-time signal processing, the device of the invention can advantageously control a device that measures and analyzes an electrocardiographic signal and/or a device for implementation of a therapy based on such an analysis, e.g., cardiac stimulation.

### BRIEF DESCRIPTION OF THE DRAWINGS

Further benefits, features and characteristics of the present invention will become apparent to a person of ordinary skill in the art in view of the following detailed description of a preferred embodiment of the invention, made with reference to the annexed drawings, in which:

FIG. 1 is a diagrammatic view of an embodiment having two sensors positioned on the thorax of a patient; and

FIG. 2 illustrates a representative phonocardiographic signal delivered by the sensors of FIG. 1 and analyzed by the device.

### DETAILED DESCRIPTION OF THE INVENTION

Broadly, the present invention concerns measuring blood pressure indirectly based upon the noises emitted by the heart.

One can recognize in each cycle of a healthy heart two major noises: (1) a first cardiac noise that corresponds to the closing of the mitral valve and incidentally of the tricuspid valve at the time of the beginning of the ventricular contraction (systole), and (2) a second cardiac noise that corresponds to the closing of the aortic valve and incidentally of the pulmonary valve, at the end of the same cardiac contraction (systole). These noises are collected (detected or sensed) by the device of the present invention by using phonocardiographic equipment, a known technique that involves placing on the thoracic wall of the patient, at about the level of the heart, a sensor. The sensor is one that can respond to the acoustic signals generated mainly by the closing of the cardiac valves and transmitted through the thorax, and can transform the sensor acoustic signals into electric signals (the so-called raw phonocardiographic signals). The sensor can be a microphone or, in alternative, an accelerometer presenting a sufficiently large band-width extending to an inaudible range, typically a band-width from about 10 to about 500 Hz.

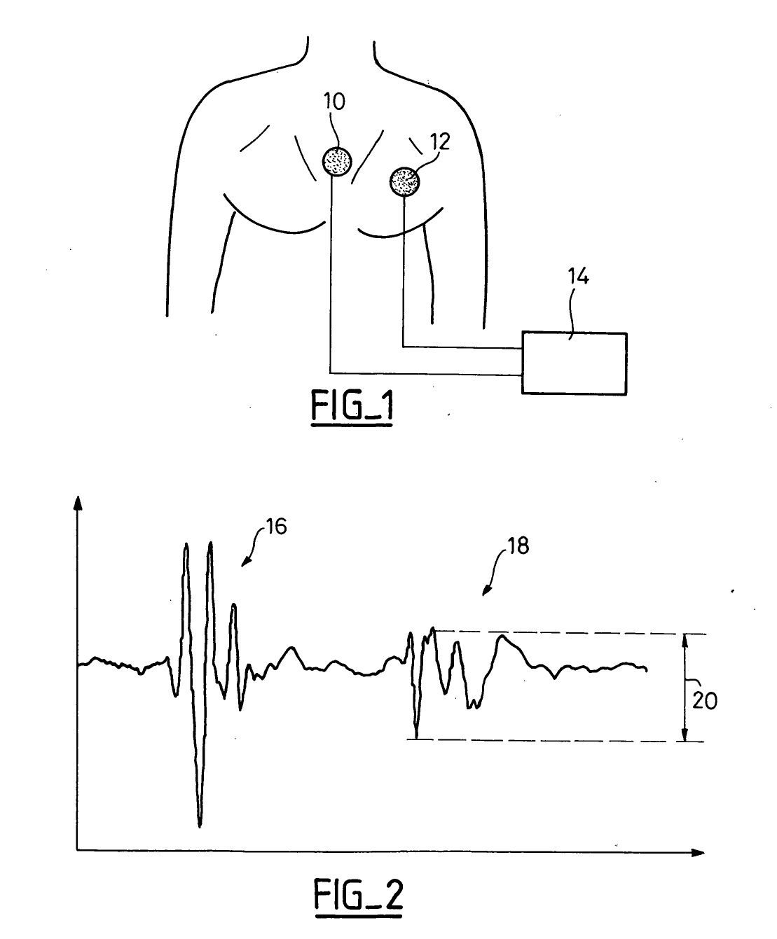

With reference to FIG. 1, a configuration is illustrated with two microphones 10, 12 spaced apart on the thorax to straddle the location where the acoustic signal amplitude has a maximum amplitude and thus receive acoustic signals of virtually identical amplitudes. The phonocardiographic signals of these multiple microphones are then combined (summed, and preferably averaged or scaled) to give in effect a stable average phonocardiographic signal that can be analyzed by autonomous portable equipment for recording and analysis. In particular, the ambulatory equipment maybe of the same type as the Holter devices used for the continuous recording of the electrocardiographic signals in an ambulatory patient.

The obtained phonocardiographic signal is then a signal presenting periodically, with each cardiac cycle, the first and second cardiac noises as indicated above, respectively illustrated as wave complexes 16 and 18.

Second cardiac noise 18 has an amplitude 20 that can vary over time. This amplitude, more specifically the difference (i.e., the extrema) measured between the maximum and minimum values of the signal amplitude during the relevant noise time period, is mainly related to the shock wave created by the closing of the aortic valves under the effect of the variation of pressure between aorta and ventricle. When the ventricle has finished its contraction, the intraventricular pressure is low, and the aortic pressure corresponds to the systolic blood pressure. It is thus possible to find a relation between the systolic blood pressure and the second noise. By analysis of the amplitude, it is thus possible to deliver a value, indicated hereafter as “phono-arterial index”, giving a relative indication of the value of the blood pressure.

In the alternative or in complement of the analysis of amplitude 20 of second cardiac noise 18, it is possible to determine the phono-arterial index based upon other measurable parameters of the second cardiac noise, such as the energy of the signal, the variation of the derivative of the signal (in particular, the maximum value of this derivative), the surface of the signal or of the principal peak of this signal, or indeed, of a combination of some or all of the foregoing parameters.

The analysis of the phono-arterial index and its recording over a relatively long duration will make it possible for a physician to perform a diagnosis, to recognize the existence of a pathology, to determine the occurrence and the importance of one or more episodes of hypotension or hypertension, etc.

In addition, in an optional embodiment, the device of the present invention, can be coupled with another device that carries out an absolute measurement of blood pressure, for example, a device implementing one of the techniques indicated above implying the inflation of a balloon placed around a member or a finger. In this way, the device of the invention ensures a continuous follow-up of the variations of the pressure and, in the event of an observed anomaly (for example, a sudden large pressure drop), it will be able to start the implementation of the absolute measurement of the pressure. This then will supplement the indications that will be provided to the physician for his diagnosis. Such a device thus reduces the risk of missing a hypertensive or hypotensive crisis that might occur between two measurements taken in the case of the prior known techniques using only a periodic inflation of a balloon at predetermined intervals. In this way, the device of the present invention detects the pressure change and in response controls the inflation of the balloon, only when the absolute measurement of the pressure is useful.

Conversely, the device of the invention can be used to inhibit a device for taking an absolute measurement of the blood pressure, for example, during periods when the pressure is particularly stable, as in sleep phases. The periodic inflation of the arm-band is thus inhibited for these periods and does not come to bother the patient during his sleep.

The device of the invention can be also advantageously coupled with a device, implanted or external, that measures and analyzes an electrocardiographic signal. Indeed, because of the excellent correlation of the phono-arterial index with the blood pressure, it is, for example, possible to detect a syncope at its beginning, well before the bradycardia that is generated by the fall of the pressure. Thus, the detection of the beginning of a syncope by the device of the invention can, for example, make it possible to start a detailed electrocardiographic recording immediately, in order to be able to have fine measurements of the rate of heartbeat and, if necessary, to implement without delay a suitable therapy. The latter event may occur by controlling the implanted prosthesis or by delivery of a medication.

A desired application of the technique of the present invention supposes a suitable collection of the cardiac noises. The collection has to always remain identical regardless of the positional changes of the patient and the variations of the patient's respiratory cycle. In particular, in the case of an ambulatory collection, the positional changes of the patient are important. The heart moves within the thorax, and the acoustic waves are not propagated in the same manner when the patient is in upright position as when lying down, etc. In this way, if one does not carry out any correction, the correlative modifications of the signal are likely to involve a bad interpretation of the blood pressure variations.

There are several manners of resolving this difficulty. A first solution, mentioned above, employs using a plurality of sensors (e.g., two or more microphones) placed on opposite sides of the site where the amplitude is a maximum, and they each receive a signal of virtually the identical amplitude. During the positional changes, the acoustic shock wave will be directed more towards one or the other of the microphones, and a simple calculation (e.g., weighting the contribution of each microphone sensor appropriately) makes it possible to obtain a perfectly stable average signal from two (or more) signals collected.

A second solution concerns employing a reference element taken from the same signal, for example, the amplitude of first cardiac noise 16, and then to standardize (normalize) the amplitude of second cardiac noise 18 according to this determined reference element. In the event of a positional change deteriorating the two components in an identical way, one will find a phono-arterial index perfectly stabilized. Measures must be taken, however, to detect modifications of the reference parameter due, for example, to a total fall of pressure caused by cardiac insufficiency. Such a fall would lead to a reduction in the amplitude of all of the components of the signal, a phenomenon that it is necessary of course to eliminate in order not to distort the determination of the phono-arterial index.

A third solution involves performing a self-learning process (i.e., an initial calibration) at the time of the installation of the microphones, namely placing the patient in several different positions and determining for each position a corresponding corrective factor. The corrective factor may be an average of a number of samples or a single value. This corrective factor will be applied later at the evaluation of the phono-arterial parameter according to the position of the patient determined at the time of measurement.

It is necessary also to take account of the respiratory movements of the patient, so that the movements do not disturb the collection of the phonocardiographic signal. The respiratory movements introduce more or less air into the lungs and constitute an absorbent. Thus the air volume modulates in a cyclic manner the amplitude of the signals collected on the thoracic wall. The time-constant of this cyclic modulation is relatively long (typically about four seconds) compared to the variations of the phonocardiographic signal. Consequently, a simple low-pass filtering makes it possible to eliminate the variations introduced by respiratory activity rather simply and effectively.

Nevertheless, if one wants to obtain a short response time for the device, it can be advantageous to apply an adaptive filtering, or even a dynamic correction of gain. Adaptive filtering can be carried out by means of a low-pass filter having a variable cut-off frequency that is calculated according to the frequency of breathing, the latter being recognized based upon the cyclic modulation of amplitude in the range of 10 to 20 cycles per minutes. Dynamic filtering, on the other hand, requires recognizing the variations of amplitude present during the respiratory cycle, and modulating the gain according to the phase within this cycle. These techniques are each in themselves well known, and will not be described more in detail.

Suitable devices for which the present invention has application include, for example, ambulatory Holter recorder and analyzer available from Ela Médical, Montrouge France. This devices are known under the trade marks Syneflash and Syneview. With respect to suitable known devices that may be used to record and treat the phonocardiographic signal, reference is made to U.S. Pat. No. 5,669,393 commonly assigned herewith to Ela Medical and incorporated herein by reference in its entity. The creation of suitable software instructions for controlling a microprocessor controlled device of the present invention to perform the aforementioned functions of the present invention are believed to be within the abilities of a person of ordinary skill in the art.

One skilled in the art will appreciate that the present invention can be practiced by other than the described embodiments, which are presented for purposes of illustration and not of limitation.

I claim:

1. A device for the noninvasive measurement of a patient's blood pressure, comprising:
at least one sensor to be placed on a thoracic wall of a patient, said sensor being responsive to acoustic signals generated by the closing of the patient's cardiac valves and transmitted through the thorax, and producing an electronic phonocardiographic signal corresponding to said detected acoustic signals;
discriminating means for identifying and extracting from the phonocardiographic signal a vibratory profile related to a second cardiac noise periodically produced at the end of the systole, and
means for analyzing at least one predetermined parameter of the vibratory profile and, in response, delivering according to said at least one parameter, a phono-arterial index value representative of the patient's blood pressure.

2. The device of claim 1, further comprising a memory and means for recording the phonocardiographic signal in said memory, wherein the discriminating means and the analyzing means further comprise means for processing the recorded phonocardiographic signal.

3. The device of claim 2, wherein said at least one parameter is a parameter selected from among the group consisting of an extrema of the phonocardiographic signal for a duration of the vibratory profile, an energy of the phonocardiographic signal, a variation of a derivative of the phonocardiographic signal, a surface of the phonocardiographic signal, and a combination of the forgoing.

4. The device of claim 2, wherein the analyzing means further comprises means for applying a weighted value to said at least one parameter, said weighted value being variable from one vibratory profile to another.

5. The device of claim 4, wherein:
the discriminating means further comprises means for identifying and extracting from the phonocardiographic signal a second vibratory profile related to a cardiac noise periodically produced at a beginning of systole, and
the analyzing means further comprises means for analyzing at least one predetermined parameter of said second vibratory profile, and producing a weighted value that is a function of said at least one predetermined parameter.

6. The device of claim 2, wherein said at least one sensor further comprises at least a first sensor and a second sensor, and means for combining the phonocardiographic signals delivered by said at least first and second sensors into a combined signal, wherein said phonocardiographic signal of said discriminating mean comprises said combined signal.

7. The device of claim 2, further comprising means for evaluating a body position of the patient, and means for determining and memorizing a plurality of average levels of the phonocardiographic signal according to a corresponding plurality of body positions, wherein the analyzing means further comprises means for identifying a body position of the patient corresponding to the detected vibratory profile, and means for applying to said at least one parameter a weighted value that is a function of the average level memorized for the body position corresponding to the identified body position at the moment of the analysis.

8. The device of claim 2, further comprising means for low-pass filtering the determined phono-arterial index.

9. The device of claim 8, further comprising means for evaluating a respiratory frequency of said patient, wherein said means for low-pass filtering further comprises means for adaptive filtering at a variable cut-off frequency, and means for adjusting said variable cut-off frequency according to the respiratory frequency at the time of the analysis.

10. The device of claim 1, further comprising means for analyzing a respiratory cycle, and means for filtering the phono-arterial index, wherein said filtering means further comprises means for dynamic filtering at a variable gain and means for selecting the gain according to the phase of the respiratory cycle at the time of the analysis.

11. The device of claim 1, further comprising a second means for measuring blood pressure, having an output corresponding to an absolute value of said blood pressure, and means for operating said second blood pressure measurement means in response to the delivered phono-arterial index.

12. The device of claim 1, further comprising means for controlling a measuring and analyzing device of an electrocardiographic signal in response to the delivered phono-arterial index.

13. The device of claim 1, further comprising means for controlling a device for implementation of a therapy in response to the delivered phono-arterial index.

14. The device of claim 1, wherein said at least one parameter is a parameter selected from among the group consisting of an extrema of the phonocardiographic signal for a duration of the vibratory profile, an energy of the phonocardiographic signal, a variation of a derivative of the phonocardiographic signal, a surface of the phonocardiographic signal, and a combination of the forgoing.

15. The device of claim 1, wherein the analyzing means further comprises means for applying a weighted value to said at least one parameter, said weighted value being variable from one vibratory profile to another.

16. The device of claim 15, wherein:
the discriminating means further comprises means for identifying and extracting from the phonocardiographic signal a second vibratory profile related to a cardiac noise periodically produced at a beginning of systole, and
the analyzing means further comprises means for analyzing at least one predetermined parameter of said second vibratory profile, and producing a weighted value that is a function of said at least one predetermined parameter.

17. The device of claim 1, wherein said at least one sensor further comprises at least a first sensor and a second sensor, and means for combining the phonocardiographic signals delivered by said at least first and second sensors into a combined signal, wherein said phonocardiographic signal of said discriminating mean comprises said combined signal.

18. The device of claim 1, further comprising means for evaluating a body position of the patient, and means for determining and memorizing a plurality of average levels of the phonocardiographic signal according to a corresponding plurality of body positions, wherein the analyzing means further comprises means for identifying a body position of the patient corresponding to the detected vibratory profile, and means for applying to said at least one parameter a weighted value that is a function of the average level memorized for the body position corresponding to the identified body position at the moment of the analysis.

19. The device of claim 1, further comprising means for low-pass filtering the determined phono-arterial index.

20. The device of claim 19, further comprising means for evaluating a respiratory frequency of said patient, wherein said means for low-pass filtering further comprises means for adaptive filtering at a variable cut-off frequency, and means for adjusting said variable cut-off frequency according to the respiratory frequency at the time of the analysis.
